(19) United States
(12) Patent Application Publication
(10) Pub. No.: US 2007/0160698 A1
(43) Pub. Date: Jul. 12, 2007

(54) Muscular strength enhancing agent

(76) Inventors: Toshiaki Waga, Ibaraki (JP); Koichi Nakazato, Tokyo (JP); HongSun Song, Tokyo (JP); Junji Inoue, Ibaraki (JP)

Correspondence Address: JACKIE JAY SCHWARTZ, 1350 Broadway, Suite 1510, NEW YORK, NY, 10018, US

(21) Appl. No.: 10/597,805

(22) PCT Filed: Feb. 9, 2005
(86) PCT No.: PCT/JP05/01884

§ 371 Date: Aug. 8, 2006

(30) Foreign Application Priority Data
Feb. 10, 2004 (JP) 2004-033905

(51) Int. Cl. A61K 36/73; A61K 31/7048; A61K 31/353

(52) U.S. Cl. 424/765; 514/27; 514/456

(57) ABSTRACT

The present invention relates to a muscular strength enhancing agent produced from fruits, and provides a strength enhancing agent or a body-fat regulating agent, derived from natural products, that may enhance muscle strength, decrease body fat or suppress body-fat accumulation without decreasing the amount of skeletal muscle or the weight of viscus; more specifically, a polyphenol produced from fruits is used to provide muscular strength enhancing agents or body-fat regulating agents, foods/beverages and pharmaceuticals that may enhance muscular strength, decrease body fat or suppress body-fat accumulation. Strength enhancing agents or body-fat regulating agents effective to enhance muscular strength, of which active component being a polyphenol derived from fruits, and foods/beverages and pharmaceuticals containing them.

### TECHNICAL FIELD

The present invention relates to muscular strength enhancing agents produced from fruits, in particular to muscular strength enhancing agents and body-fat regulating agents that may enhance muscular strength and suppress body-fat accumulation.

### BACKGROUND ART

In these years, the Japanese eat habit has become significantly westernized and become a high-calorie diet. In particular, Japanese people are likely to have excess fat accumulation due to excess lipid intake, and thus to suffer from obesity. Obesity tends to be complicated with hypertension, glucose intolerance, hyperlipemia etc. and is a risk factor in terms of ischemic heart disease, stroke, diabetes; accordingly, prevention of obesity is extremely important from the view point of lifestyle diseases prevention. In addition, obesity is undesirable in terms of appearance.

Conventionally, obesity has been prevented or treated mostly based on diet restriction or dietetic foods; however, diet restriction causes additional mental difficulties, erroneous execution thereof may lead to such risks as nutritional disorders or symptoms such as cibophobia may develop. Further, it has been found that unreasonable restriction of diets or inhibition of digestive enzymes may also decrease the amount of skeletal muscle in addition to body fat, since the nutrient components supplied to the body decrease in general, and the decrease of skeletal muscle may cause a rebound after dieting, or a decay of muscle force. Such weight control with the decrease of the skeletal muscle is undesired for sportsmen and athletes in particular since exercise performance degrades due to the resulting muscle decay. Further, athletes, who are classified by their body weight such as in judo and boxing, and body-builders usually exercise to strengthen their muscles and to enhance their muscular strength by way of the resistance training as well as a diet regimen for controlling their body weight. Such athletes must decrease their body weight or fat while increasing their muscular strength at the same time, which are difficult to accomplish at the same time, thus sufficient knowledge of nutrition is usually necessary. If they try exclusively to decrease their body weight, their entire diets are likely to be limited; however, an energy deficiency due to restricted diets or insufficient nutritive components of micronutrients often results in an overload on their bodies, possibly causing problems or breakdowns.

On the other hand, various drugs and medicines have been utilized and administered; however, much care must be taken concerning their efficiency and also their side effects such as muscle decay for example. Thus, the prevention or amelioration of obesity has not been previously attained in easy or convenient ways. From the viewpoint of the recent trend for fine health or the requirements with respect to training or weight control of athletes, natural materials have been sought which may bring about a decrease of body fat exclusively, without decreasing the amount of skeletal muscle.

Concerning natural materials, it is known that fruit polyphenols produced from fruits provide a number of medicinal benefits. For example, Patent Documents 1 and 2 disclose antioxidation properties, AEC-inhibitory activities, antimutagenic effects, hyaluronidase-inhibitory activities, histamine release-inhibitory activities, and GTase activity-inhibitory activities, odor-eliminating effects for malodorous substances and production-blocking effect for malodorous substances; Patent Document 3 discloses UV-absorbing capacity over a wide range of wavelength and free radical-eliminating performance; Patent Document 4 discloses oxidation control for in vivo lipids, amelioration of HDL-cholesterol versus total-cholesterol or control effects for absorbing food cholesterol into bodies and Patent Document 5 discloses an effect of decreasing the cholesterol content in stored meats.

As for natural materials other than those from fruits, Patent Document 6 discloses that a tamarind-testa extract containing procyanidin as the active component may inhibit a glucidase and induce a decrease of body weight. Patent Document 7 discloses that inclusion of proanthocyanidins extracted from grapes into protein foods may suppress muscular-tension decreases occurring immediately after training stimulation.

Patent Document 1: Japanese Unexamined Patent Publication No. 07-285876

Patent Document 2: Japanese Unexamined Patent Publication No. 2002-47196

Patent Document 3: Japanese Unexamined Patent Publication No. 09-175982

Patent Document 4: Japanese Unexamined Patent Publication No. 10-330278

Patent Document 5: Japanese Unexamined Patent Publication No. 11-318347

Patent Document 6: Japanese Unexamined Patent Publication No. 09-291039

Patent Document 7: Japanese Unexamined Patent Publication No. 11-75708

However, the prior art has not disclosed yet that fruit polyphenols produced from fruits may provide an action of enhancing muscular strength and an effect of suppressing body-fat accumulation, although a number of medicinal benefits are known as disclosed in Patent Documents 1 to 5. Further, the tamarind-testa extract containing procyanidin as the active ingredient disclosed in Patent Document 6 does not solely reduce body fat even though it does reduce body weight. Still further, the proanthocyanidin extracted from grapes disclosed in Patent Document 7 does not enhance muscular tension even though it does suppress muscular tension decay.

It is an object of the invention to develop a muscular strength enhancing agent and/or a body-fat regulating agent that enhances muscle tension and decreases body fat and suppress body-fat accumulation without decreasing the amount of skeletal muscle or visceral weight, in particular to provide a muscular strength enhancing agent and/or a body-fat regulating agent derived from natural products.

### DISCLOSURE OF THE INVENTION

The present inventors have researched and investigated diligently to attain the objects described above, and consequently have found that a polyphenol derived from fruits such as apples among numerous natural products may provide such effects as enhancing muscular strength and also reducing body fat and/or suppressing body-fat accumulation without decreasing the amount of skeletal muscle or visceral weight, and the present invention has completed on the basis of this knowledge.

That is, the first aspect of the present invention relates to a muscular strength enhancing agent of which the active component is a polyphenol derived from fruits.

The second aspect of the present invention relates to the muscular strength enhancing agent described in the first invention in which the fruits are apples.

The third aspect of the present invention relates to a body-fat regulating agent of which the active component is a polyphenol derived from apples.

The fourth aspect of the present invention relates to the muscular strength enhancing agent or the body-fat regulating agent described in any one of the first to the third aspects of the invention in which procyanidin is included within the polyphenol in a high content.

The fifth aspect of the present invention relates to the muscular strength enhancing agent or the body-fat regulating agent described in any one of the first to the forth aspects of the invention in which the polyphenol contains a simple polyphenol compound and a polymer polyphenol compound in a higher content.

The sixth aspect of the present invention relates to the muscular strength enhancing agent or the body-fat regulating agent described in the fifth aspect of the invention in which the simple polyphenol compound is selected from the group of caffeic acid derivatives, p-coumaric acid derivatives, flavan-3-ols, flavonols and dihydrochalcones.

The seventh aspect of the present invention relates to the muscular strength enhancing agent or the body-fat regulating agent described in the fifth aspect of the invention in which the polymer polyphenol compound is selected from condensed tannins.

The eighth aspect of the present invention relates to the muscular strength enhancing agent or the body-fat regulating agent described in any one of the first to the seventh aspects of the invention in which the muscle is skeletal muscle.

The ninth aspect of the present invention relates to the muscular strength enhancing agent or the body-fat regulating agent described in any one of the third to the seventh aspects of the invention in which the body fat is visceral fat.

The tenth aspect of the present invention relates to the muscular strength enhancing agent or the body-fat regulating agent described in any one of the third to the seventh aspects of the invention in which the body fat is subcutaneous fat.

The eleventh aspect of the present invention relates to foods/beverages that contain the muscular strength enhancing agent or the body-fat regulating agent described in any one of the first to the tenth aspects of the invention.

The twelfth aspect of the present invention relates to pharmaceuticals that contain the muscular strength enhancing agent or the body-fat regulating agent described in any one of the first to the tenth aspects of the invention.

The thirteenth aspects of the present invention relates to a use of the polyphenol derived from fruits for producing a muscular strength enhancing agent.

The fourteenth aspect of the present invention relates a use of the polyphenol derived from apple for producing a body-fat regulating agent.

The “polyphenol derived from fruits” in the present invention (hereinafter referred to as “fruit polyphenol”) may be produced, for example, by squeezing fruits to form a juice, clarifying the juice into a clear liquid, passing the liquid through an absorbing synthetic resin made of styrene-divinylbenzene polymer, then washing the synthetic resin with water to remove sufficiently saccharides and organic acids followed by extracting with hydroscopic ethanol. The resulting fruit polyphenol may act as a muscular strength enhancing substance, and as such it may be added into foods/beverages for preventing muscle-decay or strengthening muscle-force or utilized as therapeutic agents for treating muscle-decay, or formulated into pharmaceuticals. In addition, it may act to decrease body fat, to suppress body-fat accumulation, thus it may provide such effects as weight control, prevention of obesity-induced is deceases, and health maintenance.

Since the fruit polyphenol is generally contained in fruits, particularly unripe apples in higher amounts, it is preferred to utilize unripe apples for producing the fruit polyphenol. The term “unripe” represents fruits prior to being commercially available in markets. Such unripe fruits have usually been discarded since they are not valuable as articles, therefore their utilization may lead to an effective utilization of resources.

We have confirmed that the fruit polyphenol according to the present invention mainly contains simple polyphenol compounds including caffeic acid derivatives, p-coumaric acid derivatives, flavan-3-ols (catechins), flavonols (quercetin glycosides), dihydrochalcones (phloretin glycosides) etc. and polymer polyphenol compounds including condensed tannins (polymeric procyanidin formed of two to four polymerized catechins) as its composition.

The term “enhance muscular strength” means that the amount of muscle is increased, or that the muscular tension (force displayed by the muscles) is enhanced. Accordingly, the muscular strength enhancing agent of the present invention encompasses those acting to enhance muscular tetanic-tension and twitch-tension or to increase muscle amount i.e. muscular substance. The term “muscle decay” means that the amount of muscles is increased or the muscular strength is weakened, and encompasses amyotrophy, decrease of muscle mass and muscle fatigue. Specific examples thereof include, but are not limited to, amyotrophy in elderly persons, amyotrophy due to inactivity of resting under treatment in the case of orthopedic deceases, accidents or disorders, amyotrophy induced under weightless conditions such as an outer space, and muscle fatigue induced under specific pressure conditions such as at the sea bed.

The muscular strength enhancing agent according to the present invention may be applied to any kind of muscles such as skeletal, smooth and cardiac muscles, and preferably it is applied to skeletal muscle in particular. The “skeletal muscle” encompasses facial, masticatory, neck, pectoral, abdominal, back, upper limb and lower limb muscles.

The muscular strength enhancing agent according to the present invention may decrease body fat as well as suppress body-fat accumulation, thus the body fat in particular visceral fat may be decreased, thereby symptoms such as obesity and hyperlipidemia may be ameliorated or prevented. As such, it is beneficial to prevent obesity-induced deceases or to maintain healthy bodies.

The term “body-fat regulating agent” indicates those agents that may suppress the accumulation of excess absorbed energy as body fat, those that promote the conversion of excess energy into the energy for activating muscles or viscus, or those that to lower excess accumulated body fat, thereby controlling and suppressing the amount of body fat. Accordingly, the regulation of body fat according to the present invention may prevent the accumulation of body fat, without unduly repressing the absorption of energy or becoming dangerously thin due to the decrease of body fat. Such advantageous effects of the present invention have been demonstrated from the fact that a group, which was fed with high-nutrition food while being administered the body-fat regulating agent of the present invention, cleared a fat decrease in terms of body fat and furthermore the amount of skeletal muscle and visceral weight were not decreased, although the average body weight was substantially the same as that of the group to which the body-fat regulating agent of the present invention was not administered, as shown Examples below. Accordingly, when the body-fat regulating agent of the present invention is administered during a stage when the body weight is increasing, it may act to suppress fat accumulation and to prevent obesity, and when administered under the conditions of obesity, it may ameliorate the obesity by action of promoting the conversion of the fat into protein such as muscle.

The muscular strength enhancing agent according to the present invention may be formulated along with conventional carriers, adjuvants, additives and the like into drugs for oral administration etc. by conventional processes, and may be utilized as pharmaceuticals, alternatively may mixed with food or beverage materials to prepare foods/beverages.

The administration thereof as pharmaceuticals may treat decayed muscle to enhance the strength, and intake thereof from health foods or functional food may be available for enhancing muscular strength, preventing obesity-induced is diseases, health maintenance and the like.

Since the muscular strength enhancing agent according to the present invention contains fruit polyphenol derived from natural materials as the active component, it provides living bodies with less side effects and higher safety. Further, it may have an excellent effect of decreasing the accumulated body fat or suppressing the accumulation of excessive energy in the form of body fat, thus muscular strength may be enhanced along with a decrease in body fat and a reduction in body weight. Consequently, foods/beverages or pharmaceuticals including the agent may be taken for a long period with higher safety, and may be extremely effective to prevent or ameliorate muscle decay, to prevent or ameliorate obesity, and also to enhance muscular strength in the training of sportsmen or athletes.

### BRIEF DESCRIPTION OF THE DRAWINGS

FIG. 1 is a graph showing the feeding period versus the body weight.

FIG. 2 is a graph showing the feeding days versus the total intake fed till the feeding days.

FIG. 3 is a graph showing the muscle tetanic-tension generated by the gastrocnemius of rats measured before the feeding and at three weeks after the initial intake.

FIG. 4 is a graph showing the muscle tetanic-tension per weight of the gastrocnemius of rats at three weeks after the initial intake.

FIG. 5 is a graph showing the muscle twitch-tension generated by the gastrocnemius of rats versus the elapsed time at three weeks after the initial intake.

FIG. 6 is a graph showing the weights of the lower limb skeletal muscle and visceral tissues of rats at three weeks after the initial intake.

FIG. 7 is a graph showing the amounts of visceral fat of rats at three weeks after the initial intake.

### PREFERRED MODE FOR CARRYING OUT THE INVENTION

The fruits utilized in the present invention as the raw material belong to the rose family, and specifically are apples, pears, and peaches etc., preferably apples. The fruits may be mature or unripe; it is preferred that the fruits are unripe in particular since they contain higher amount of polyphenol compounds and also a great deal of various active ingredients that have a wide variety of physiologic activities.

Concerning the process for squeezing and making juice from fruits, for example, raw material is washed, then fractured and pressed to form the squeezed juice with or without adding sulfurous acid, preferably with adding a pectic enzyme. Then the intermediate is centrifuged and filtered to prepare a clear juice. Concerning the extraction process, the washed raw material is mixed with an alcohol such as ethanol and methanol, and fractured, then extracted while being immersed and crushed or heated and refluxed. Then the intermediate is concentrated under reduced pressure to evaporate the alcohol, centrifuged and filtered, or divided by use of organic solvents such as hexane and chloroform and filtered, thereby to produce a clear liquid of the extract.

Concerning the process for purifying the resultant clear juice or clear extracted liquid, the clear juice or clear extracted liquid is passed through a column that is filled with an absorbent, capable of selectively absorbing and being eluted polyphenols, such as synthetic adsorptive styrene-divinylbenzene resins, anion-exchange resins, and silica gel to which octadecyl group is chemically attached (ODS), thereby the polyphenol fraction is adsorbed within the column. Then distilled water is passed through it to wash, followed by passing 20-100% hydroscopic alcohol (e.g. ethanol) solution, preferably, about 50% hydroscopic alcohol solution through the column, thereby the polyphenol fraction can be eluted and recovered. The resulting polyphenol solution is concentrated under reduced pressure to evaporate the alcohol to produce a concentrated liquid of fruit polyphenol. Further, the concentrated liquid may be spray-dried or freeze-dried with or without adding powder adjuvants such as dextrin to produce fruit polyphenol powder formulations.

The composition of the fruit polyphenol according to the present invention mainly contains simple polyphenol compounds including caffeic acid derivatives, p-coumaric acid derivatives, flavan-3-ols (catechins), flavonols (quercetin glycosides), dihydrochalcones (phloretin glycosides) etc. and polymer polyphenol compounds including condensed tannins (polymeric procyanidin formed of two to four polymerized catechins). These ingredients are effective to enhance muscular strength as well as to decrease body fat or to suppress accumulation thereof.

The polyphenol extracted from fruits such as apples, may be formulated along with conventional carriers, adjuvants, additives and the like into drugs for oral etc. by conventional processes, and may be utilized as pharmaceuticals, or alternatively may mixed with food or beverage materials to prepare foods/beverages.

The pharmaceuticals may be produced in a dosage form of tablets, capsules, granules, syrups or the like. When these products are administered to human bodies, the dosage depends on the kind of formulation, the administrator process conditions, the symptoms of the subjects, physical conditions, heights, body weights and the like; usually the dosage is effective in an amount of 0.01 to 1000 mg/kg-body, preferably 0.1 to 80 mg/kg-body for one to several times per day.

The pharmaceuticals containing the muscular strength enhancing agent with body-fat regulating ability of the present invention may have conventional dosage forms such as tabellae, capsules, sugar-coated pills, pills, tablets, subtle granules, aerosols, syrups, emulsions, suspensions and liquids through conventional processes using inactive, nontoxic, pharmaceutically acceptable excipients or solvents. The respective compounds effective for treatment may be present in an amount of about 0.5 to 90 weight % based on the entire composition, that is, in an amount sufficient for achieving the effects described above. The formulation may be prepared, for example, by diluting the active compounds with solvents and/or excipients, or with emulsifiers and/or suspending agents if appropriate. When water is employed as a diluent, an organic solvent may also be employed as an adjuvant solvent if appropriate. Examples of the adjuvants include water; nontoxic organic solvents such as paraffin e.g. petroleum distillate fraction; vegetable oils such as peanut oils and seeds oils; alcohols such as ethanol and methanol; excipients; powdered natural minerals such as clay, alumina, talc and chalk; powdered synthetic minerals such as highly dispersed silica and silicate; saccharides such as sucrose, lactose and dextrose; emulsifiers such as polyoxyethylene fatty acid ester and polyoxyethylene fatty alcohol ether, alkylsulfonate and arylsulfonate; suspending agents such as lignin sulfurous acid waste liquid, methylcellulose, starch and polyvinyl pyrrolidone; and lubricants such as magnesium stearate, talc, stearic acid and sodium lauryl sulfate.

The administration may be carried out in conventional ways, preferably by an oral route, or may be administered parenterally. In particular cases, the administration may be carried out sublingually or intravenously. The injecting medium may be aqueous solutions that contain conventional stabilizers, solubilizing agents and/or buffer solutions. These additional agents may be, for example, borate buffers, ethanol, dimethysulfoxide, complexings agents (e.g. ethylenediaminetetra acetic acid), viscosity-adjusting polymers (e.g. liquid polyethylene oxide) or polyethylene derivatives of hydrogenated sorbitan. In the case of oral administration, and when aqueous dispersants are employed in particular, favoring agents or colorants may be added to the active components flavoring along with the adjuvant agents described above.

The foods/beverages containing the inventive muscular strength enhancing agent with body-fat regulating ability may be produced in the dosage forms described above; preferably they are produced in food forms such as dry foods, semiliquid foods, gel foods, drinks and the like, more specifically, cold beverages, teas, coffees, soups, liqueurs, low-malt beers, milks, lactoserum drinks, lactic acid bacteria beverages, jelly drinks, sweets such as candies, chewing gums, oleasters, yogurt, ice cream, rice crackers, cookies and the like, utilizing conventional basic materials. The raw materials for foods may also be processed in conventional ways into these food forms with the addition of predetermined amounts of the inventive muscular strength enhancing agent. The content thereof may be determined depending on the properties of the various foods/beverages, intake, safety, cost and the like, and usually is 0.01 to 50 weight %, preferably 0.1 to 10 weight %; the compounding thereof may be properly carried out at a suitable production step depending on the purpose.

The foods/beverages containing the inventive muscular strength enhancing agent may be made available for enhancing muscular strength, preventing diseases, health maintenance and the like; they are usually taken as processed foods containing them in an amount of 0.1 to 1000 g, preferably 1 to 100 g per day, but are not limited to this.

When the inventive muscular strength enhancing agent with body-fat regulating ability is added to foods/beverages, the muscular strength enhancing agent with body-fat regulating ability may be added directly as the powder form, preferably is added as an aqueous solution, aqueous alcohol solution or alcohol solution containing 1 to 2% of the muscular strength enhancing agent.

In addition, the foods/beverages containing the inventive muscular strength enhancing agent may be compounded with various ingredients depending on the food forms.

Examples of the various ingredients described above include starch, cornstarch, dextrin, sucralose, glucose, fructose, malt sugar, stevioside, corn syrup, lactose, nicotinic-acid amide, calcium pantothenate, calcium salts, vitamin B family compounds, aspartame, xylitol, sorbitol, sorbitan fatty acid ester, L-ascorbic acid, alpha-tocopherol, sodium erythorbate, citric acid, tartaric acid, malic acid, succinic acid, lactic acid, gum arabic, carrageenan, pectin, amino acids, yeast extra, glycerin fatty acid ester, sucrose fatty acid ester, glycerin, propylene glycol, casein, gelatin, agar, dyes, flavors, preservatives and the like.

The pharmaceuticals and foods/beverages containing the inventive muscular strength enhancing agent with body-fat regulating ability may enhance muscular strength and reduce accumulation of body fat, in particular to enhance muscular strength of skeletal muscle and to decrease visceral fat, therefore they may be beneficial to prevent or treat obesity without decreasing the amount of skeletal muscle or visceral weight, furthermore they may be extremely advantageous for enhancing muscle force in the training of sportsmen or athletes.

### EXAMPLES

The present invention will be explained in more detail with reference to examples in the following, but the present invention is not limited by these examples.

### Example 1

Preparation of Muscular Strength Enhancing Agent from Fruits

An amount of 1000 kg of unripe apples were washed and fragmented by use of a hammer crusher or hammer mill fragmenting machine, while adding a potassium disulfide antioxidant in an amount of 600 ppm. The fragmented fruits were squeezed by use of a belt-press squeezing machine. Then 48,000 units of pectic enzyme of pectinase was added to the resulting 800 L of juice (60 units/L), and the mixture was allowed to stand overnight at 40 to 50° C. to produce a clarified juice. The resulting juice was centrifuged to remove the solid content thereby to raise the clarity still further.

Then the juice was directed through a column filled with a styrene-divinylbenzen adsorbing resin (trade name: Sepabeads SP-850 from Mitsubishi Chemical Co.). After the juice was passed through completely, an amount of deionized water of one to two times the column volume was passed through to wash the column, then 50 to 60 volume % of ethanol in an amount of one to two times the column volume was passed through the column to elute the fruit polyphenol adsorbed to the resin, thereby to yield 24 L of concentrated fruit polyphenol liquid having a solid content of 20 w/v %. The concentrated liquid was spray-dried by a spray-dryer to form a fruit polyphenol formulation in an amount of 3.4 kg.

### Test Example 1

Effect of Enhancing the Muscular Strength

The muscular strength enhancing agent obtained in Example 1 was examined with respect to the effect of enhancing the muscular tetanic-tension using 11 week old Wistar male rats.

Subjects, Food, Feeding Process

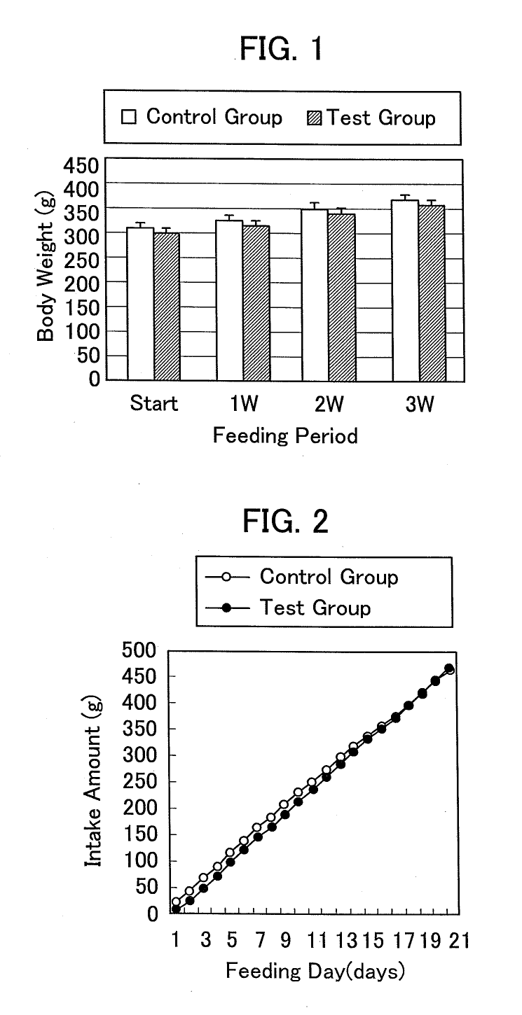

Twenty-four 11 week old Wistar male rats were fed preliminarily for one week; the non-abnormal rats therefrom were divided into two groups such that the total body weights of each group were the same. The rats of the first group were fed for three weeks while allowing to free access food and water, in which the food was prepared by sufficiently blending an experimental animal diet (from Oriental Yeast Co., Ltd.) with the muscular strength enhancing agent obtained in Example 1 at 5 weight %, and the body weights of the rats were measured over time. In parallel, the rats of the second group, which was the control group, were fed solely with the experimental animal diet described above in the same way for three weeks, and the body weights of the rats were measured likewise over time. The body weights were measured and compared between the test and control groups at the initial stage, one week, two weeks, and three weeks after the start, as shown in FIG. 1. The amount of food taken by the rats during the feeding period is shown in FIG. 2 for the two groups. The composition of the experimental animal diet (from Oriental Yeast Co., Ltd.) was based on the standard purified diet AIN-93M announced by the American Institution of Nutrition as shown in Table 1.

TABLE 1
Composition of Diet AIN-93M

| Ingredient | content % |
|---|---|
| casein | 14.0 |
| cornstarch | 62.1 |
| sucrose | 10.0 |
| cellulose | 5.0 |
| soybean oil | 4.0 |
| t-butylhydroquinone | 0.0008 |
| mineral mixture | 3.5 |
| vitamin mixture | 1.0 |
| L-cystine | 0.18 |
| choline bitartarate | 0.25 |
| calorie (cal/g) | 341.2 |

Examination of Muscle Tetanic-Tension

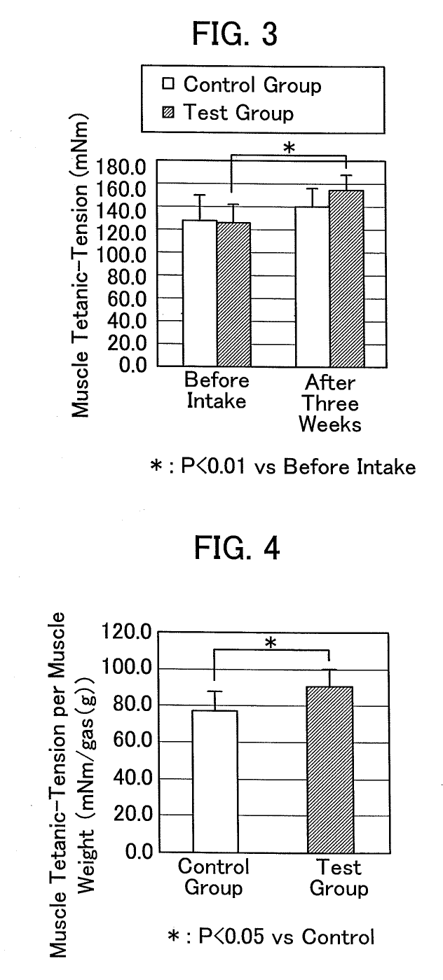

The rats of the test and control groups after three weeks from the feeding initiation were tested with respect to the strength generated by the gastrocnemius of the rats by measuring the ankle isometric exertion torque as described below. The results are shown in FIG. 3 in comparison with the control group. The muscular tetanic-tension per weight of the gastrocnemius is shown in FIG. 4 for the stage after three weeks.

The results show that the strength generated by gastrocnemius tends to increase at three weeks after the feeding initiation, in particular the test group which had taken the muscular strength enhancing agent combined with the feeding diet had a remarkably enhanced muscle tetanic-tension as shown in FIG. 3. Furthermore, the muscle tetanic-tension per weight of the gastrocnemius was enhanced about 20% compared to the control group as shown in FIG. 4. Accordingly, it is demonstrated that the muscular strength enhancing agent containing polyphenol derived from fruits may exhibit a remarkable effect in enhancing muscle strength. Method for Measuring the Ankle Isometric Exertion Torque

A rat is set in a prone position and the chest portion is fixed to a fixing table, when the hip joint and knee joint were disposed in an extended position, the lower limb was arranged in a horizontal position, and the ankle joint was allowed to be free. Then, the revolving center of a pedal-shaped torque meter and the revolving center of the ankle joint of the rat were adjusted to coincide with each other, and also the plantar parts were arranged to contact the pedals, thus the rat test animal and the measuring device were fixed completely. The employed torque meter was confirmed to be linear in a range of 0.1 mNm to 100 mNm.

After the rat was fixed to the fixing table and the measuring device, a skin electrode (Bitload, from Nihon Kohden Co.) was attached to the skin at directly above the distal portion of the inside of gastrocnemius, then a tetany was induced by applying an electrical excitation of 100 Hz and 15 V from an electrical excitation device (SEN-3301, from Nihon Kohden Co.). The frequency and voltage at the electrical excitation were selected to cause the maximum exertion muscle force. The exertion torque was input into a personal computer through a high-speed data collecting device (PowerLab, from AD Instruments Co.).

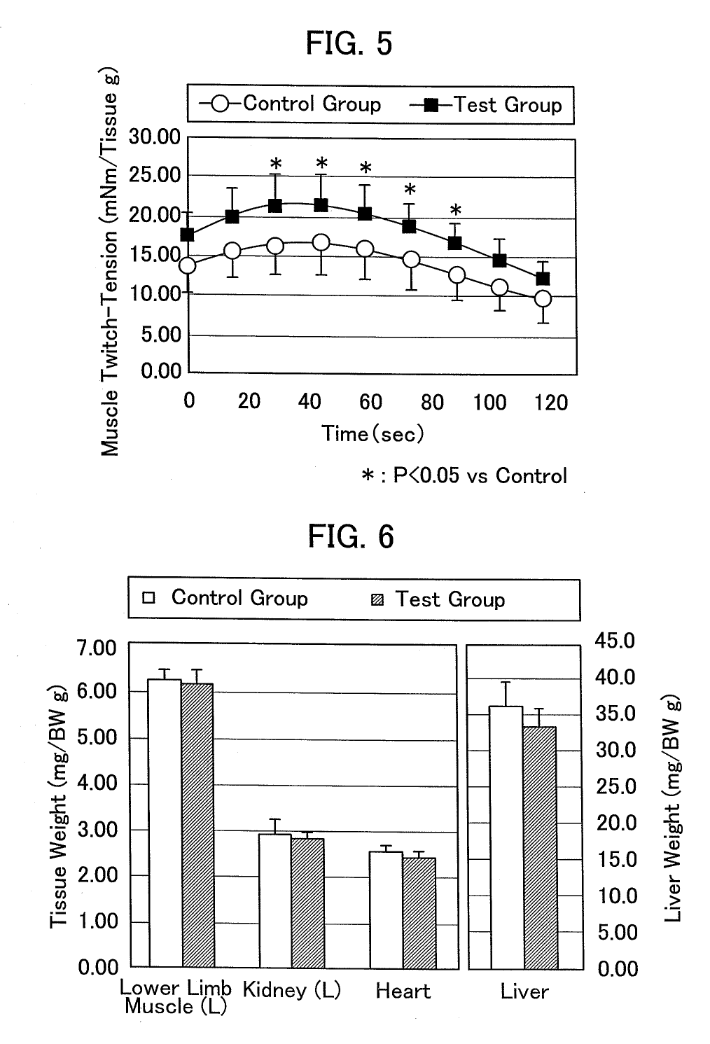

The rats of the test and control groups after three weeks from the feeding initiation were measured in terms of the exertion torque through intermittently inducing a gastrocnemius twitch of 120 times over 2 minutes by applying an electrical excitation every one second in accordance with the measuring method described below; the results are shown in FIG. 5 with respect to the exertion property of the gastrocnemius in comparison to the control group.

The results demonstrate that the muscular twich-tension of the test group is higher than that of the control group, and a significant difference appeared during 30 seconds to 90 seconds in particular as shown in FIG. 5.

Method for Measuring Changes of the Ankle Isometric Exertion Torque over Time

A rat was fixed in the similar way as the measurement of the isometric exertion torque described above, and a skin electrode was attached to the rat. Then an electrical excitation of 1 Hz and 15 V was applied to cause twitches intermittently every one second for 2 minutes (120 times) through the electrical excitation device (SEN-3301, from Nihon Kohden Co.). The exertion torque was input into a personal computer through a high-speed data collecting device (PowerLab, from AD Instruments Co.).

### Test Example 2

Effect to Suppress Body-Fat Accumulation

Examination of Tissue Weight

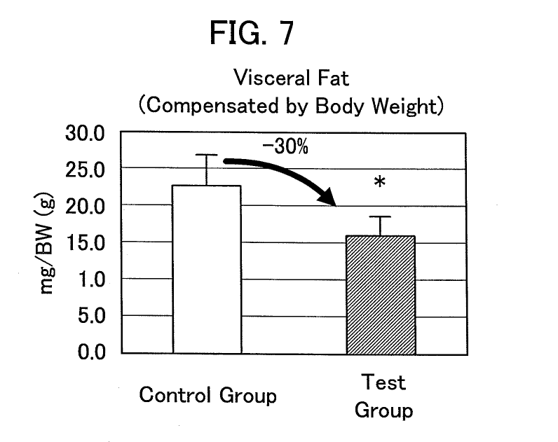

The rats of the test and control groups at three weeks after the initial feeding of Test Example 1 were measured for the weight of the heart, liver, kidney, viscus, soleus, plantaris, and gastrocnemius after these were extirpated. The results are shown in Table 2 and FIG. 6 in comparison with the control group. The weights of tissues and visceral fat in Table 2, FIGS. 6 and 7 are represented in terms of values compensated by body weight after dissection.

TABLE 2
Tissue Weight

Tissue Weight(compensated
by body weight) (mg/BWg)

| Tissue | Test Group | Control Group |
|---|---|---|
| gastrocnemius | 4.83 ± 0.27 | 4.89 ± 0.16 |
| soleus | 0.40 ± 0.02 | 0.40 ± 0.03 |
| plantaris | 1.00 ± 0.06 | 1.02 ± 0.04 |
| kidney | 2.86 ± 0.15 | 2.94 ± 0.29 |
| heart | 2.45 ± 0.15 | 2.54 ± 0.17 |
| liver | 33.6 ± 2.8 | 36.6 ± 3.1 |
| visceral fat | 15.8 ± 2.7* | 22.6 ± 4.1 |

*P < 0.01 vs control group

From the results, the changes of body weight and intake amount were not recognized to be different between the test group and the control group, as shown in FIGS. 1 and 2. The respective tissue weights compensated by body weight after the dissection were also not different between the test group and the control group in terms of the lower limb skeletal muscle (i.e. soleus, plantaris gastrocnemius), liver, heart and kidney as shown in Table 2, FIGS. 6 and 7; however, the amount of visceral fat of the test group was about 30% less than that of the control group (see FIG. 7). These facts demonstrate that the muscular strength enhancing agent containing polyphenol derived from fruits may suppress body-fat accumulation without decreasing the amount of skeletal muscle and visceral weight; that is, it may have the effect of rendering the form of stored energy into muscle or the like, rather than fat.

### Example 2

Tablet, Capsule

A total of 100 g, consisting of 55.5 g of the muscular strength enhancing agent obtained in Example 1, 41.0 g of crystalline cellulose, 2.0 g of silicone dioxide fine particles and 1.5 g of sucrose fatty ester were mixed uniformly, then formed into tablets and capsules by a conventional method.

### Example 3

Powdered Drug, Granules

A total of 100 g, consisting of 20.0 g of the muscular strength enhancing agent obtained in Example 1, 30.0 g of starch and 50.0 g of lactose were mixed uniformly, then formed into a powdered drug and granules by a conventional method.

### Example 4

Beverage

A beverage was prepared from 0.45 g of the muscular strength enhancing agent obtained in Example 1, 15.0 g of a clear concentrated apple juice, 5.0 g of a fruit sugar, 0.2 g of citric acid, 2.0 g of a flavor, 0.15 g of a dye, 0.05 g of sodium ascorbate and 77.15 ml of water.

### Example 5

Candy

Using a total of 100 g, consisting of 20.0 g of sucrose, 70.0 g of starch syrup (solid content 75%), 9.5 g of water, 0.45 g of a colorant, 0.04 g of a flavor and 0.01 g of the muscular strength enhancing agent obtained in Example 1, a candy was prepared in accordance with a conventional process.

### Example 6

Cookie

Using a total of 100 g, consisting of 32.0 g of soft powder, 16.0 g of whole egg, 19.0 g of butter, 25.0 g of sugar, 7.2 g of water, 0.2 g of baking powder and 0.6 g of the muscular strength enhancing agent obtained in Example 1, a cookie was prepared in accordance with a conventional process.

1. A muscular strength enhancing agent comprising an active component which is a polyphenol derived from a fruit.

2. The muscular strength enhancing agent according to claim 1, wherein the fruit is an apple.

3. A body-fat regulating agent comprising an active component is a polyphenol derived from an apple.

4. The muscular strength enhancing agent according to claim 1, wherein procyanidin is included within the poyphenol in a high content.

5. The muscular strength enhancing agent according to claim 1, wherein the polyphenol contains a monomer polyphenol compound and an oligomer-polymer polyphenol compound in a higher content.

6. The muscular strength enhancing agent according to claim 5, wherein the monomer polyphenol compound is selected from caffeic acid derivatives, p-coumaric acid derivatives, flavan-3-ols, flavonols and dihydrochalcones.

7. The muscular strength enhancing agent according to claim 5, wherein the oligomer-polymer polyphenol compound is selected from condensed tannins.

8. The muscular strength enhancing agent according to claim 1, wherein the muscle is skeletal muscle.

9. The body-fat regulating agent according claim 3, wherein the body fat is visceral fat.

10. Or the body-fat regulating agent according claim 3, wherein the body fat is subcutaneous fat.

11. A food or a beverage comprising the muscular strength enhancing agent according to claim 1.

12. A pharmaceutical which contains the muscular strength enhancing agent according to claim 1.

13. Use of a polyphenol derived from fruits for producing a muscular strength enhancing agent.

14. Use of a polyphenol derived from an apple for producing a body-fat regulating agent.

15. The body-fat regulating agent according to claim 3, wherein proycyanidin is included within the polyphenol in a high content.

16. The body-fat regulating agent according to claim 3, wherein the polyphenol contains a monomer polyphenol compound and an oligomer-polymer polyphenol compound in a higher continent.

17. The body-fat regulating agent according to claim 16, wherein the monomer polyphenol compound is selected from caffeic acid derivatives, p-coumaric acid derivatives, flavan-3ols, flavonols and dihydrochalcones.

18. The body-fat regulating agent according to claim 16, wherein the oligomer-polymer polyphenol compound is selected from condensed tannins.

19. A food or a beverage comprising the body fat regulating agent according to claim 3.

20. A pharmaceutical which contains the body-fat regulating agent according to claim 3.
